(19) United States
(12) Patent Application Publication
(10) Pub. No.: US 2011/0172405 A1
(43) Pub. Date: Jul. 14, 2011

(54) METHOD FOR SMALL RNA ISOLATION

(75) Inventors: Rohini Dhulipala, Amherst, NY (US); Yuyang Christine Cai, Cranbury, NJ (US); Miao Jiang, Cedar Knolls, NJ (US); Mubasher Dar, North Brunswick, NJ (US)

(73) Assignee: GE HEALTHCARE BIO-SCIENCES CORP., PISCATAWAY, NJ (US)

(21) Appl. No.: 13/063,546

(22) PCT Filed: Sep. 17, 2009
(86) PCT No.: PCT/US09/57233

§ 371 Date: Mar. 11, 2011

Related U.S. Application Data
(60) Provisional application No. 61/097,604, filed on Sep. 17, 2008.
(60) Provisional application No. 61/148,126, filed on Jan. 29, 2009.

(51) Int. Cl. C07H 21/02

(52) U.S. Cl. 536/23.1

(57) ABSTRACT

This invention relates to a simple and rapid method for the extraction and purification of small RNA from a sample solution. Accordingly, a sample is first mixed with an organic solvent to form a mixture containing the solvent. The mixture is applied to a first mineral support for large RNA to bind. The filtrate is collected which contain unbound small RNA, and is mixed with a second organic solvent to form a second mixture containing the second solvent. This second mixture is applied to a second mineral support for small RNA to bind. After a wash step, the small RNA is eluted. Also provided is a method for the isolation of large RNA, by eluting the large RNA from the first mineral support. In addition, total protein is present in the filtrate and can be isolated by a conventional method.

### CROSS-REFERENCE TO RELATED APPLICATIONS

This application is a filing under 35 U.S.C. §371 and claims priority to international patent application number PCT/US2009/057233 filed Sep. 17, 2009, published on Mar. 25, 2010 as WO 2010/033652, which claims priority to U.S. provisional patent application Nos. 61/097,604 filed Sep. 17, 2008 and 61/148,126 filed Jan. 29, 2009; the disclosures of which are incorporated herein by reference in their entireties.

### FIELD OF THE INVENTION

This invention relates to methods for the isolation of nucleic acids. More specifically, it relates to a simple and rapid method for the extraction and purification of small RNA and total RNA.

### BACKGROUND OF THE INVENTION

The last three decades has seen considerable effort in the development of improved methods for the isolation and purification of nucleic acids and proteins from biological sources. This has been due mainly to the increasing applications of nucleic acids and proteins in the medical and biological sciences. Genomic DNA isolated from blood, tissue or cultured cells has several applications, which include PCR, sequencing, genotyping, comparative genomic hybridization and Southern Blotting. Plasmid DNA has been utilized in sequencing, PCR, in the development of vaccines and in gene therapy. Isolated RNA has a variety of downstream applications, including in vitro translation, cDNA synthesis, RT-PCR and for microarray gene expression analysis. In the protein field, identification of proteins by Western Blotting and 2D-electrophoresis have become important tools in studying gene expression in disease research and basic research and identification of specific proteins for diagnostic purposes, as exemplified by viral protein detection.

The analysis and in vitro manipulation of nucleic acids and proteins is typically preceded by an isolation step in order to free the samples from unwanted contaminants, which may interfere with subsequent processing procedures. For the vast majority of procedures in research and diagnostic molecular biology, extracted nucleic acids and proteins are required as the first step.

The increased use of RNA, DNA and proteins has created a need for fast, simple and reliable methods and reagents for isolating DNA, RNA and proteins. In many applications, collecting the biological material sample and subsequent analysis thereof would be substantially simplified if the three cellular components (RNA, DNA and proteins) could be simultaneously isolated from a single sample. The simultaneous isolation is especially important when the sample size is so small, such as in biopsy, that it precludes its separation into smaller samples to perform separate isolation protocols for DNA, RNA and proteins.

Epigenetics includes study of DNA and protein modifications (methylation, acetylation, phosphorylation, etc) and protein DNA interactions controlling expression of genes and subsequent effects on cellular biology. Small regulatory RNAs such as micro RNA and siRNA are also known to regulate gene expression by epigenetic mechanisms. For example, chromatin is modified by these modification events, altering its structure, thereby allowing expression of genes. The interplay between chromatin modification and microRNAs expression is expected to gain a pivotal role in providing diagnostics and therapy in various cancers. Thus the availability of an efficient miRNA isolation method is expected to be a core component for epigenetic analysis.

Further, microRNAs (miRNA) regulate gene expression and dysregulation of miRNA have been implicated in a number of diseases or conditions. If microRNA can be isolated from the same sample, together with total protein, genomic DNA and total RNA (i.e., large RNA containing mRNA), there is a clear advantage to our understanding of the interaction and effects among them. An effective means for the isolation of microRNA would also aid the development of microRNA-based diagnostics and therapeutics, in the fields of cancer, neurology, and cardiology among others.

Purification of miRNA traditionally relied on organic extraction followed by alcohol precipitation. This time consuming method results in loss of much of the small RNA, such as miRNA, from the RNA population and is therefore inefficient. Several companies have developed miRNA isolation kits based on organic extraction followed by simple binding and purification of small RNA on a silica fiber matrix using specialized binding and wash solutions, e.g., MIRVANA™ (Ambion); miRNeasy (Qiagen); microRNA Purification Kit (Norgen). These kits provide moderately high yields of all small RNA (under 200 nucleotides long, down to about 10 nucleotides long) from a variety of sample sources including cells and tissue types.

A novel and advantageous method for the purification of small RNA is presented here. This method can be further expanded to allow the simultaneous isolation of small RNA with one or more of total RNA, genomic DNA, and total protein from the same sample.

### SUMMARY OF THE INVENTION

In general, the instant invention provides a simple and rapid method for the extraction and purification of small RNA (including microRNA) from a sample solution, such as a biological sample lysate. In addition, large RNA in excess of 200 nucleotides in length is separated from the small RNA and can be isolated as well.

In one embodiment for the isolation of small RNA, a sample is first mixed with an organic solvent to form a mixture. The mixture is applied to a first mineral support for large RNA to bind. The filtrate is collected and mixed with a second organic solvent to form a second mixture. This second mixture is applied to a second mineral support for small RNA to bind. After a wash step, the small RNA is eluted. When the sample is a biological lysate, it is preferable that the sample is lysed using a lysis solution that includes a chaotropic salt, non-ionic detergent and reducing agent. For the ease of operation, the first and the second mineral support are usually the same material. Preferably, they are each silica membrane columns A preferred first and second organic solvent are dipolar aprotic solvents. A most preferred organic solvent is acetone. It is found that at a lower solvent concentration, large RNA binds to the mineral support while small RNA does not. At increased concentration of the solvent, small RNA would bind and thus separated from other contaminants in the sample. It is discovered that the use of acetone not only allows for selective purification of small RNA, the yield of small RNA is also increased than prior art methods.

In a variation of the embodiment, large RNA bound on the first mineral support can be isolated as well. Thus, the first mineral support is washed and the large RNA is eluted.

Further, filtrate off the second mineral support contains total protein from the sample. Thus, total protein can be isolated from the filtrate by any conventional method for protein isolation.

In another variation of the embodiment, a biological lysate, prior to forming a mixture with the first solvent, is subjected to a phenol chloroform extraction step. This removes most large genomic DNA and proteins, thus improving the purity of the isolated small and large RNA.

In another embodiment, it is provided compositions and kits for isolation of the small RNA as well as large RNA using the various workflows.

The above and further features and advantages of the instant invention will become clearer from the following detailed description and claims.

### BRIEF DESCRIPTION OF THE DRAWINGS

FIG. 1 presents a schematic diagram of an embodiment of the invention (Workflow-1) for the isolation of enriched total RNA and small RNAs (micro RNAs) from a single sample with phenol chloroform extraction step after sample lysis.

FIG. 2 presents a schematic diagram of an embodiment of the invention (Workflow-2) for the isolation of enriched small RNAs (micro RNAs) with phenol chloroform extraction step after sample lysis.

FIG. 3 presents a schematic diagram of an embodiment of the invention (Workflow-3) for the isolation of enriched total RNA and small RNAs (micro RNAs) from a single sample without phenol chloroform extraction step after sample lysis.

FIG. 4 presents a schematic diagram of an embodiment of the invention (Workflow-4) for the isolation of enriched small RNAs (micro RNAs) without phenol chloroform extraction step after sample lysis. By eliminating phenol-chloroform separation steps as shown in work flow-4 significantly reduces the protocol time small RNA isolation.

FIG. 5 shows gel images of total RNA and small RNAs (micro RNA) isolated using Acetone according to certain embodiments of the invention. Increase the amount of Acetone increased small RNA yields.

FIG. 6 shows feasibility experiment results obtained using workflow-1. Results indicate that protocol described in workflow-1 successfully isolates enriched total RNA and small RNAs including micro RNAs.

FIG. 7 shows that small RNA can be successfully isolated without compromising quality or yield with a shorter protocol according to workflow-2.

FIG. 8 shows results obtained from qRT-PCR graph for four microRNA, confirming the presence of both low and high copy number microRNA in the isolated small RNA sample according to an embodiment of the invention.

### DETAILED DESCRIPTION OF THE INVENTION

In its broadest aspects, the invention encompasses a method for isolating substantially pure and undegraded small RNA from a sample solution. Accordingly, a sample is first mixed with an organic solvent to form a mixture containing the solvent. The mixture is applied to a first mineral support for large RNA to bind. The flowthrough (filtrate) is collected which contains unbound small RNA, and mixed with a second organic solvent to form a second mixture containing the second solvent. This second mixture is applied to a second mineral support for small RNA to bind. After one or two wash steps, the small RNA is eluted.

By small RNA, it is generally meant in this disclosure to include RNA molecules of less than 200 nucleotides in length. These include a variety of different RNA species, such as tRNA, rRNA, but more importantly small regulatory RNA such as microRNA. In contrast, RNA molecules of greater than 200 nucleotides generally bind to the first mineral support and thus separated from the small RNA. This latter group of RNA molecules is herein referred to, interchangeably, as big RNA, large RNA, or total RNA.

It is discovered that in the presence of certain organic solvents, RNA binds to the mineral support. Further, RNA molecules of different length respond differently to the concentration of the organic solvent. Thus, at a lower concentration of the organic solvent, only large RNA molecules bind to the mineral support, while at a higher concentration, smaller RNA binds to the mineral support as well.

As an example, polar protic solvents such as lower aliphatic alcohol are suitable organic solvents. Preferably the organic solvents are dipolar aprotic solvents. Suitable dipolar aprotic solvents include but are not limited to Acetone, Tetrahydrofuran (THF), Methyl ethyl Ketone, Acetonitrile, N,N-Dimethylformamide (DMF), and Dimethyl Sulfoxide (DMSO). Preferably, the organic solvent is Acetone or Acetonitrile. Most preferably, the organic solvent is Acetone.

The first and second organic solvent can be the same or different. As an example, acetone is used in the experimental section for illustration purposes only. When acetone is used, it is determined that the preferred concentration for binding large RNA is about 35%, while the preferred concentration for binding small RNA is about 50%. It is envisioned that the first and second organic solvent do not have to be the same kind. Further, the concentration of organic solvent needed for binding of large or small RNA will vary based the nature of the solvent. However, specific detailed can be readily obtained following teachings of the current disclosure.

The sample solution from which small RNA is isolated can be any aqueous sample containing small RNA. As an example, the sample solution is RNA sample purified using conventional method. Another example is a lysate of a biological sample or biological material. The term “biological material” or “biological sample” is used in a broad sense and is intended to include a variety of biological sources that contain nucleic acids and proteins. Such sources include, without limitation, whole tissues, including biopsy materials and aspirates; in vitro cultured cells, including primary and secondary cells, transformed cell lines, and tissue and blood cells; and body fluids such as urine, sputum, semen, secretions, eye washes and aspirates, lung washes and aspirates. Fungal and plant tissues, such as leaves, roots, stems, and caps, are also within the scope of the present invention. Microorganisms and viruses that may be present on or in a biological sample are within the scope of the invention. Bacterial cells are also within the scope of the invention.

The biological sample or cells are lysed in an aqueous lysis system containing chaotropic substances and/or other salts by, in the simplest case, adding it to the cells. The term “chaotrope” or “chaotropic salt,” as used herein, refers to a substance that causes disorder in a protein or nucleic acid by, for example, but not limited to, altering the secondary, tertiary, or quaternary structure of a protein or a nucleic acid while leaving the primary structure intact. Exemplary chaotropes include, but are not limited to, Guanidine Hydrochloride, Guanidinium Thiocyanate, Sodium Thiocyanate, Sodium Iodide, Sodium Perchlorate, and Urea. A typical anionic chaotropic series, shown in order of decreasing chaotropic strength, includes: CCl₃COO⁻→CNS⁻→CF₃COO⁻→ClO₄⁻>I⁻→CH₃COO⁻→Br^{-s}, Cl⁻, or CHO₂⁻.

Some of the starting biological samples mentioned cannot be lysed directly in aqueous systems containing chaotropic substances, such as bacteria, for instance, due to the condition of their cell walls. Therefore, these starting materials must be pretreated, for example, with lytic enzymes, prior to being used in the process according to the invention.

One of the most important aspects in the isolation of RNA is to prevent degradation during the isolation procedure. Therefore, the current reagents for lysing the biological samples are preferably solutions containing large amounts of chaotropic ions. This lysis buffer immediately inactivates virtually all enzymes, preventing the enzymatic degradation of RNA. The lysis solution contains chaotropic substances in concentrations of from 0.1 to 10 M, such as from 1 to 10 M. As said chaotropic substances, there may be used, in particular, salts, such as Sodium Perchlorate, Guanidinium Chloride, Guanidinium Isothiocyanate/Guanidinium Thiocyanate, Sodium Iodide, Potassium Iodide, and/or combinations thereof.

Preferably, the lysis solution also includes a reducing agent which facilitates denaturization of RNase by the chaotropes and aids in the isolation of undegraded RNA. Preferably, the reducing agent is 2-Aminoethanethiol, tris-Carboxyethylphosphine (TCEP), or β-Mercaptoethanol.

Optionally, the lysis solution also includes a non-ionic surfactant (i.e., detergent). The presence of the detergent enables selective binding of genomic DNA to the mineral support. Exemplary nonionic surfactants include, but are not limited to, t-Octylphenoxypolyethoxyethanol (TRITON X-100™), (octylphenoxy)Polyethoxyethanol (IGEPAL™ CA-630/NP-40), Triethyleneglycol Monolauryl Ether (BRIJ™ 30), Sorbitari Monolaurate (SPAN™ 20), or the Polysorbate family of chemicals, such as Polysorbate 20 (i.e., TWEEN™ 20). Other commercially available Polysorbates include TWEEN™ 40, TWEEN™ 60 and TWEEN™ 80 (Sigma-Aldrich, St. Louis, Mo.). Any of these and other related chemicals is effective as a replacement of TWEEN™ 20.

An effective amount of non-ionic detergent for selective binding of RNA could vary slightly among the different detergents. However, the optimal concentration for each detergent (or combination of detergents) can be easily identified by some simple experiments. In general, it is discovered that a final concentration of detergent at 0.5% or greater is effective for binding. In certain embodiments, the effective concentration is between 0.5% and about 10%. In a preferred embodiment, the concentration is between 1% and 8%. It is also noted that more than one non-ionic detergent can be combined, as long as the combined concentration of the detergents is within the range of 0.5% to about 10%.

In a preferred embodiment, the lysis solution includes NP-40 (IGEPAL™ CA-630). In a most preferred embodiment, the lysis solution includes Guanidine HCl, TWEEN™ 20, NP-40 and β-Mercaptoethanol.

The lysis solution of the present invention preferably also contains a sufficient amount of buffer to maintain the pH of the solution. The pH should be maintained in the range of about 5-8. The preferred buffers for use in the lysis solution include tris-(hydroxymethyl)Aminomethane Hydrochloride (Tris-HCl), Sodium Phosphate, Sodium Acetate, Sodium Tetraborate-boric Acid and Glycine-sodium Hydroxide.

The first and second mineral support preferably consists of porous or non-porous metal oxides or mixed metal oxides, silica gel, silica membrane, materials predominantly consisting of glass, such as unmodified glass particles, powdered glass, Quartz, Alumina, Zeolites, Titanium Dioxide, Zirconium Dioxide. The particle size of the mineral support material ranges from 0.1 μm to 1000 μm, and the pore size from 2 to 1000 μm. The porous or non-porous support material may be present in the form of loose packings or may be embodied in the form of filter layers made of glass, quartz or ceramics, and/or a membrane in which silica gel is arranged, and/or particles or fibers made of mineral supports and fabrics of quartz or glass wool, as well as latex particles with or without functional groups, or frit materials made of Polyethylene, Polypropylene, Polyvinylidene Fluoride, especially ultra high molecular weight polyethylene, high density polyethylene.

The mineral support may be present in loose packing, fixed between two means, or in the form of membranes which are arranged within the hollow body of a column. Preferably, the first mineral support and the second mineral support are each silica membranes.

It is discovered that the large RNA adsorbed on the first mineral support and small RNA adsorbed on the second mineral support can both be eluted under conditions of low ionic strength or with water. Thus, an additional aspect of the invention provides a method for the separation and isolation of both large RNA and small RNA from the same sample.

After separation of the mineral support from the first or second liquid mixture, the mineral support containing bound RNA is preferably washed prior to elution of the respective large or small RNA. A wash buffer containing a high concentration of organic solvents such as lower aliphatic alcohol can be used for washing both the first and the second mineral support, to remove components other than the desired RNA.

Well-know methods are used for the separation of the mineral support material from that of the sample flowthrough (filtrate), as well as for the wash and elution steps. A simple centrifugation step is used in the examples presented below. However, methods for the separation of aqueous solution from a mineral support are well known. These include, in addition to centrifugation, vacuum or gravity based separation methods. A skilled person can readily choose a mixture of different methods based on the particular needs of the experiment or the step, giving special consideration to the need of throughput and ease of operation.

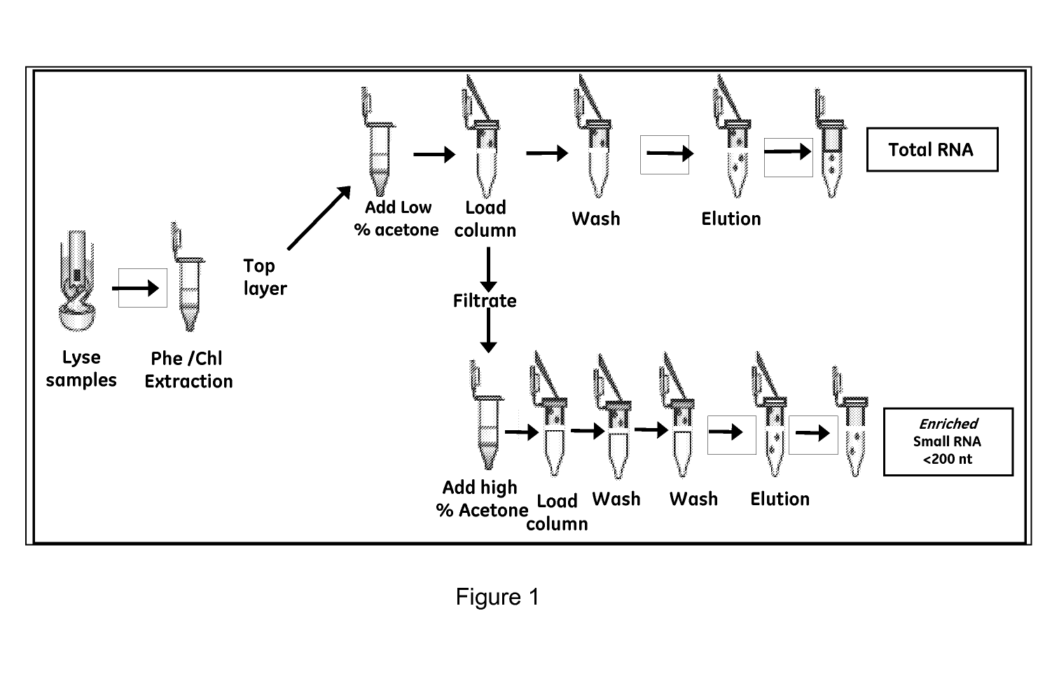

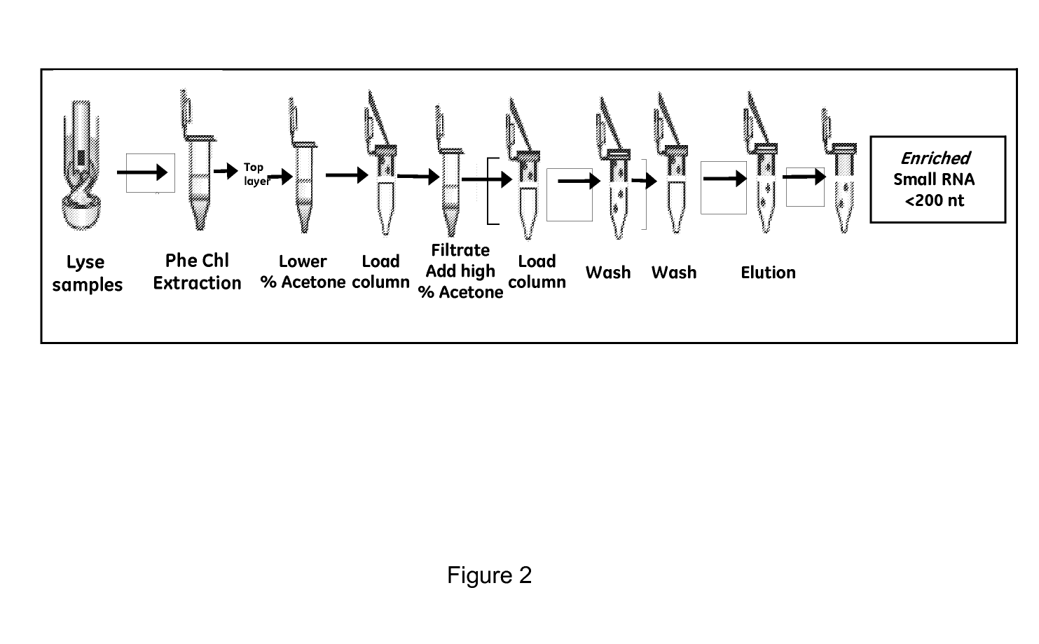

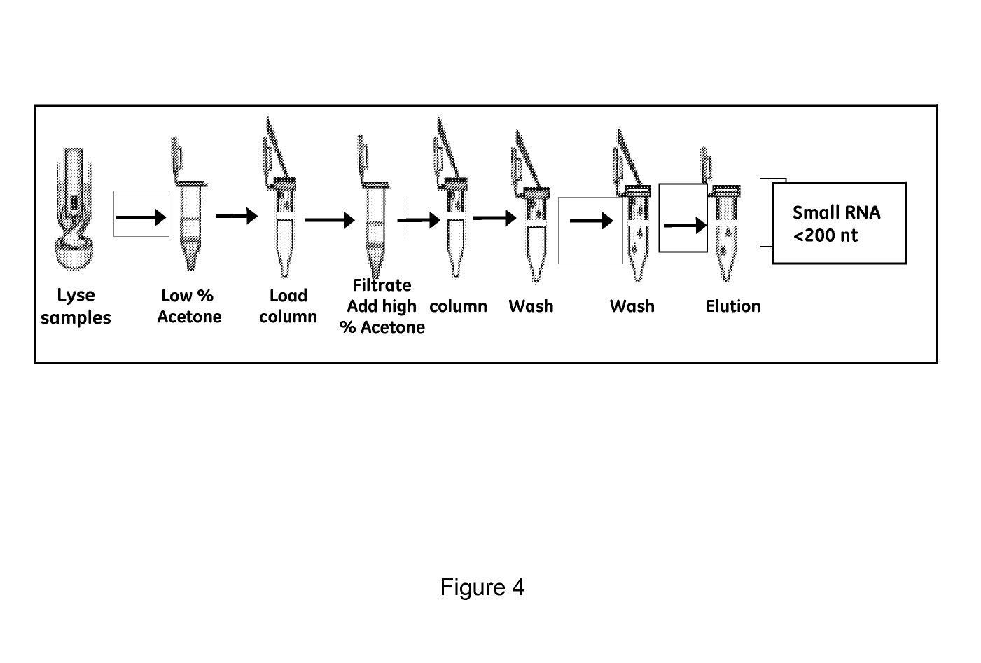

Various aspects of the invention are presented in FIGS. 1 to 4. Thus, one aspect of the invention relates to the isolation of small RNA from a sample solution as presented in workflow-4 (FIG. 4) and described above. Another aspect of the invention is presented in workflow-2 (FIG. 2). Here, a phenol chloroform extraction step is included prior to the loading of the aqueous sample onto the first mineral support. This extraction step removes large genomic DNA as well protein contaminates in the sample solution, and improves the purity of the isolated small RNA. It is important to note that the yield of small RNA by these workflows is higher than that obtained using conventional commercial protocols.

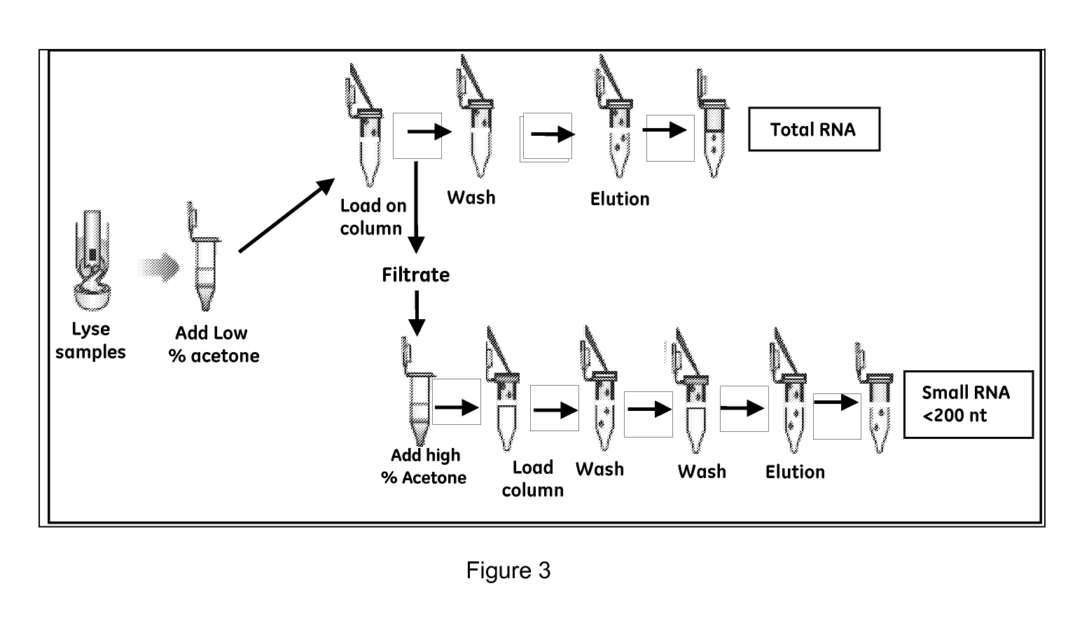

The third and fourth aspects of the invention are presented as workflows one (FIG. 1) and three (FIG. 3), respectfully. Essentially, these aspects encompass methods for isolating substantially pure and undegraded large RNA as well as small RNA from biological material and sample. The large RNA and small RNA are eluted from the first and second mineral support, respectively.

Following these protocols, large RNA and small RNA can be isolated utilizing the reagents and methods in as little as 30 minutes. These results are substantially faster than existing methods for the isolation of individual RNA components.

Following workflows one and two and the experimental conditions as further described below, large RNA and small RNA have been successfully purified from biological samples. High quality RNAs are obtained from these experiments when compared to current commercially kits, with an increased yield, suitable for use in downstream applications such as QPCR and microarray experiments.

The invention also provides a method for the isolation of total proteins with the RNA. In this regards, in workflow three and four, the filtrate (flowthrough) from the second mineral support contains total protein from the sample solution (e.g., biological sample). The total protein can be readily isolated using conventional methods, such as TCA precipitation.

Also provided are compositions and kits for the separation and/or purification of large RNA and small RNA from a biological sample. The kit comprises: a lysis solution for lysing the biological sample; a first mineral support for binding the large RNA; a second mineral support for small RNA; an elution solution for eluting large RNA from the first mineral support; an elution solution for eluting small RNA from the second mineral support, and an organic solvent such as Acetone. Optionally, the kit also includes means for isolating proteins from the flowthrough after small RNA binds to the second mineral support, as well as a user manual.

Preferably, the lysis solution in the kit includes a chaotropic salt, a non-ionic detergent and a reducing agent. Most preferably, the lysis solution includes Guanidine HCl, TWEEN™ 20, NP-40 and β-Mercaptoethanol.

Other features and advantages of the invention will be apparent from the following examples and from the claims.

### EXAMPLES

The following examples serve to illustrate the process for the isolation of total RNA and small RNA according to embodiments of the present invention and are not intended to be limiting.

### Reagents and Protocols

TABLE 1
Reagents and kits used

| Description | Composition or source |
|---|---|
| Lysis buffer | 7M GuHCl/30 mM Tris HCl/2% |
| | TWEEN ™ and 5% NP 40 |
| Wash buffer | 10 mM Tris-HCl pH 7.0 and 1 mM |
| | EDTA pH 8.0 with 80% Ethanol in |
| | the final buffer |
| RNA Elution Buffer | Water (Nuclease free) |
| gDNA Elution buffer | 10 mM Tris, 0.5 mM EDTA, pH 8.0 |
| Silica membrane spin | |
| column | |
| Absolute Acetone | Sigma Molecular biology grade |
| Absolute Ethanol | Sigma Molecular biology grade |
| 2-Mercaptoethanol | Sigma Molecular biology grade |
| (14.3M or >98%) | |
| Acid Phenol:Chlorofom | Ambion |
| miRNeasy Mini Kit | Qiagen |
| RNEASY ® Mini Kit | Qiagen |

### 1. Sample Disruption and Homogenization

### Cultured Cell Disruption and Homogenization:

a. Pellet 1×10⁶ cultured cells in a 1.5 ml microcentrifuge tube by centrifugation at 8000×g for 1 minute.
b. Completely remove the supernatant by aspiration.
c. Add 350 μl of Lysis buffer (containing B-ME).
d. Vortex to resuspend the cell pellet.
e. Homogenize the lysate by passing it through a 20-gauge needle fitted to an RNase and DNase-free syringe for at least 5 times.
f. Proceed to next step.
   Tissue Disruption and Homogenization using the POLYTRON® Homogenizer (Kinematica AG, Switzerland):
a. Place 10 mg tissue in a suitable sized tube.
b. Add 350 μl of Lysis buffer (containing βME).
c. Homogenize the tissue according to the POLYTRON®'s user manual.
d. Visually inspect the prepared homogenate and ensure thorough homogenization.
e. Proceed to step 2 or 3 (with Phenol/Chloroform step) or 4 or 5 (with out Phenol/Chloroform step)
   2. Protocol for Workflow-1 to Isolate Enriched Total RNA and Small RNA (micro RNAs) from Same Sample with Phenol: Chloroform

### 2.1 RNA Binding

a. Add 350 μl of Acid phenol: chloroform to 350 μl of homogenate in a 1.5 ml micro centrifuge tube, shake vigorously for 15 sec.
b. Centrifuge at 12,000×g for 15 minutes at +4° C.
c. Transfer the aqueous layer (upper layer) to new 1.5 ml micro centrifuge tube.
d. Add 350 μl of 70% Acetone. Mix well by pipetting up and down several times.
e. Place a new spin column in a new collection tube.
f. Transfer the entire mixture to the column.
g. Centrifuge at 8,000×g for 30 sec.
h. Save the flow-through for small RNA isolation.
i. Transfer the column to a new 2 ml collection tube.

### 2.2 Column Wash

a. Add 500 μl of Wash Buffer to the column
b. Centrifuge at 11,000×g for 1 min.
c. Discard flow through.
d. Transfer the column to an RNase free 1.5 ml micro centrifuge tube.

### 2.3 Total RNA Elution

a. Add 100 μl of Elution buffer to the center of the column.
b. Centrifuge at 11,000×g for 1 minute.
c. Discard the column and store the tube containing pure RNA at −80° C. until needed.
2.4. Small RNA (micro RNAs) isolation
a. Place a new spin column in a new collection tube.
b. To the flow through from step 2.1.h add 450 μl of 100% Acetone and mix well.
c. Mix well by pipetting up and down several times. Transfer the entire mixture to the column.
d. Centrifuge at 8,000×g for 30 sec.
e. Discard flow through.
f. Add 500 μl of wash buffer, centrifuge at 8,000×g for 30 sec.
g. Add 500 μl 80% Acetone, centrifuge at 8,000×g for 2 mins.
h. Transfer the column to an RNase free 1.5 ml micro centrifuge tube.

### 2.5 Small RNA (Micro RNAs) Elution.

a. Add 15 μl of Nuclease free water.
b. Centrifuge at 8,000×g for 1 min.
c. Collect the flow through containing the small RNA.
3. Protocol for Workflow 2 to Isolate Enriched Small RNA (Micro RNAs)

The protocol is similar to the protocol above for workflow-1. The only exception is that steps 2.1.i, 2.2 and 2.3 are not performed.

4. Protocol for Work Flow-3 the Isolation of Total RNA and Small RNA (Micro RNAs) from Same Sample without Phenol:Chloform

### 4.1 RNA Binding

a. Add 350 μl of 70% Acetone to 350 μl of lysed homogenate in a 1.5 ml micro centrifuge tubes. Mix well by pipetting up and down several times.
b. Place a new spin column in a new collection tube.
c. Transfer the entire mixture to the column.
d. Centrifuge at 8,000×g for 30 sec.
e. Save the flow-through for small RNA isolation.
f. Transfer the column to a new 2 ml collection tube.

### 4.2 Column Wash

a. Add 500 μl of Wash Buffer to the column
b. Centrifuge at 11,000×g for 1 min.
c. Discard flow through.
d. Transfer the column to an RNase free 1.5 ml micro centrifuge tube.

### 4.3 Total RNA Elution

a. Add 100 μl of Elution buffer to the center of the column.
b. Centrifuge at 11,000×g for 1 minute.
c. Discard the column and store the tube containing pure RNA at −80° C. until needed.
4.4 Small RNA (micro RNAs) isolation
a. Place a new spin column in a new collection tube.
b. To the flow through from step 4.1.e, add 450 μl of 100% Acetone and mix well by pipetting up and down several times.
c. Transfer the entire mixture to the column.
d. Centrifuge at 8,000×g for 30 sec.
e. Discard flow through.
f. Add 500 μl of wash buffer to the spin column, centrifuge at 8,000×g for 30 sec.
g. Add 500 μl 80% Acetone centrifuge at 8,000×g for 2 mins.
h. Transfer the column to an RNase free 1.5 ml microcentrifuge tube.

### 4.5 Small RNA (Micro RNAs) Elution

a. Add 15 μl of Nuclease free water.
b. Centrifuge at 8,000×g for 1 min.
c. Collect the flow through containing the small RNA.
5. Protocol for Work Flow-4 to Isolate Enriched Small RNA (Micro RNAs) from the Sample without Phenol:Chloroform

The protocol is similar to the protocol above for workflow-3. The only exception is that steps 4.1.f, 4.2 and 4.3 are not performed.

### Example 1

### RNA Isolation Using Acetone

Experiments were conducted to investigate whether acetone can be used for purification of miRNA and to identify optimal Acetone concentration in the purification of total RNA and miRNA.

The tissue samples are from rat liver tissue, while the cell cultured cells are from cultured HeLa cells. The samples were disrupted and homogenized according to the above standard protocols. After lysing the samples, the lysates were processed for the purification of total RNA and small RNA. First, a series of 350 μl-homogenized lysates were each loaded onto a silica membrane spin column. After spinning at 11,000×g for 1 minute, the flowthrough was collected for RNA isolation (the spin column contains genomic DNA which could be eluted using TE buffer). Add 250 μl, 300 μl, 350 μl or 400 μl of Absolute Acetone to the flowthrough respectively. Add 350 μl of absolute ethanol to another one as control. Mix well by pipetting up and down several times. Place new spin columns in new collection tubes. Transfer each mixture to a spin column and spin for 1 minute at 11,000×g. Discard the flow through which contains protein that could be further purified. Transfer the column to a new Collection tube, add 500 μl of Wash buffer to the column and spin for 1 minute at 11,000×g. Discard the collection tube and its contents flowthrough. Place the spin column in a new collection tube. Add 100 μl Elution buffer (dd H₂O) to the center of the column and spin for 1 minute at 11,000×g to collect the purified total RNA. Store purified RNA at −80° C.

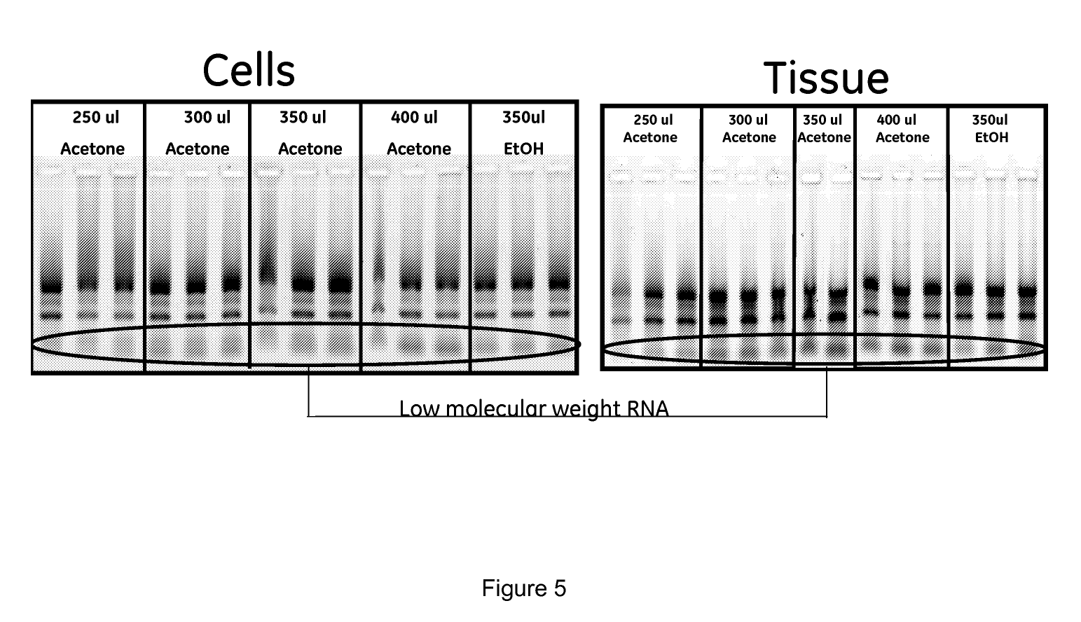

Purified total RNA was analyzed on 0.8% agarose gel. As evident in FIG. 5, the RNA preparation indicates the presence of small RNA molecules along with large RNA. Increase the amount of Acetone increased the small RNA yield. The quality and quantity of RNA and small RNA isolated using Acetone appears to be similar or better than standard ethanol precipitation.

### Example 2

### Isolation of Total and Small RNA Using Workflow-1

Feasibility experiments were carried out using workflow-1 as described earlier, with rat liver sample. Control experiments were carried out using ethanol or commercially available RNA isolation kit (Mini RNEASY® Kit for isolation total RNA and miRNeasy Mini Kit for isolation small RNAs (micro RNA), both from Qiagen).

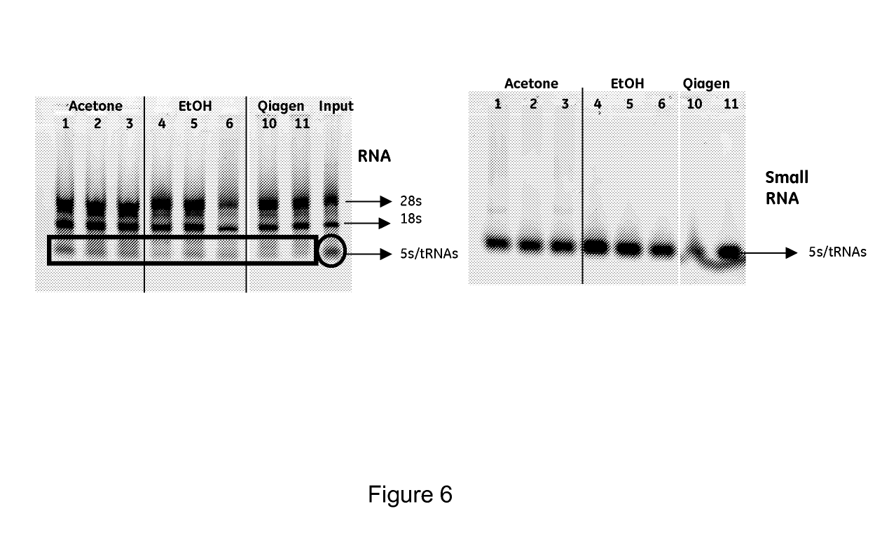

Isolated RNA (big and small) samples were run on 0.8% agarose gel. Results indicate that the protocol described in workflow-1 successfully isolates small RNA including micro RNA (FIG. 6).

### Example 3

### Isolation of Small RNA Using Workflow-2

The isolation of small RNA without larger RNA reduces total number of steps (compare workflow-2 to workflow-1). After lysis of the samples (rat liver) in Lysis buffer, lysates were processed for the purification of small RNA (micro RNAs) using protocol of workflow-2, control experiments were also carried out using commercially available RNA isolation kit (miRNeasy Mini kit, Qiagen).

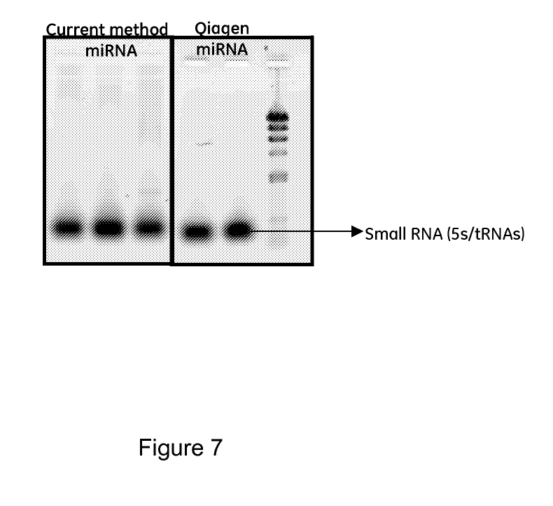

Isolated small RNA (micro RNA) samples were run on 0.8% agarose gel. The results show that using shorter protocol according to Workflow-2, enriched small RNA can be successfully isolated without compromising quality or yield (FIG. 7). Further, by eliminating phenol-chloroform extraction step as shown in workflow-3 & 4, the processing time can be significantly reduced for total RNA and small RNA isolation. In addition, the small RNA yield is higher compared to commercial kit which uses ethanol instead of acetone (Table 2).

TABLE 2
Yield of small RNA using workflow-1
compared with commercial product.

| Description | Average Yield (n = 3) |
|---|---|
| Work flow-1 | 4.4 |
| Mini RNEASY ® | 3.9 |

### Example 4

### Confirmation of the Presence of MicroRNA in Isolated Small RNA Samples

To verify that the small RNA isolated contains microRNA, we performed micro RNA specific qRT-PCR assay using four different microRNA of varying copy numbers.

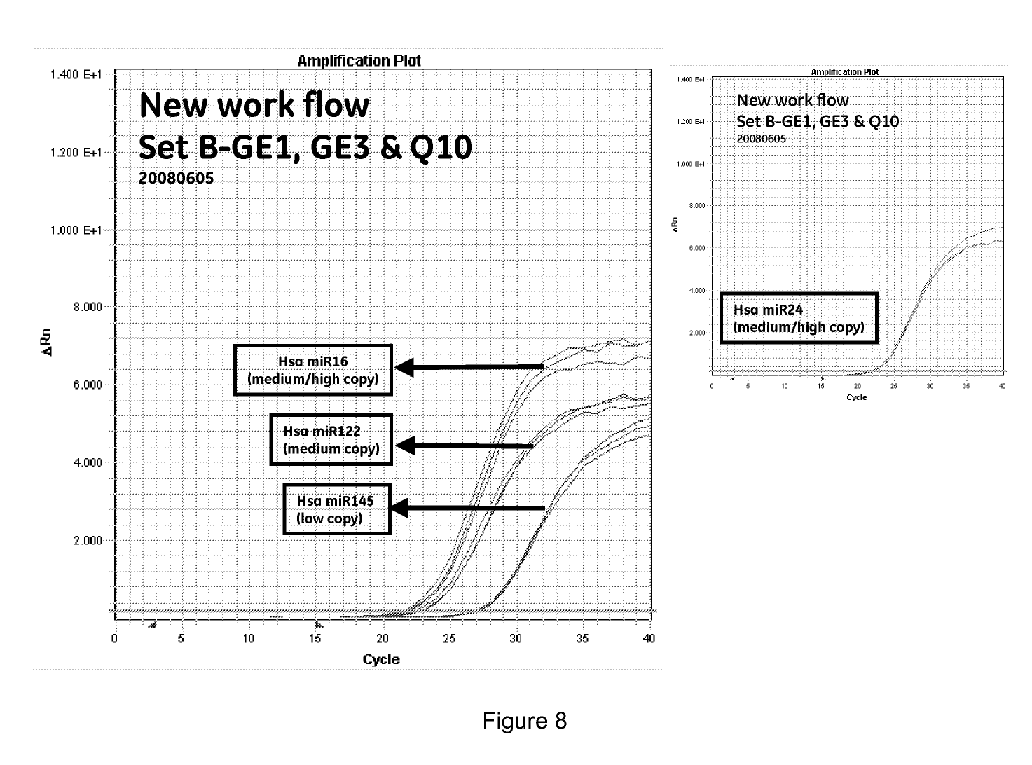

Four different micro RNA primer sets with different expression level (Low, Medium and Medium high) were utilized for the qRT-PCR analysis (Applied Biosystems Inc). We tested samples isolated form workflow-1 and the Qiagen miRNeasy kit. We were successful in detecting all four microRNA in the sample (FIG. 8).

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method for isolating small RNA from an aqueous sample solution, which method comprising:
a) mixing said aqueous sample with a first dipolar aprotic solvent to form a mixture containing a lower percent of said first solvent;
b) applying said mixture to a first mineral support under conditions such that large RNA of greater than 200 nt binds to the first mineral support;
c) collecting the flowthrough which contains small RNA;
d) mixing said flowthrough from step (d) with a second dipolar aprotic solvent to form a second mixture containing a higher percent of said second solvent;
e) applying said mixture to a second mineral support for small RNA to bind;
f) washing said second mineral support with a wash buffer; and
g) eluting said small RNA from said second mineral support.

2. The method of claim 1, wherein said aqueous sample is generated by lysing a biological sample with a lysis solution;

3. The method of claim 2, wherein said lysis solution includes chaotropic salt, non-ionic detergent and reducing agent.

4. The method of claim 3, wherein said chaotropic salt is Guanidine HCl.

5. The method of claim 3, wherein said non-ionic detergent is selected from Triethyleneglycol Monolauryl Ether, (octylphenoxy)Polyethoxyethanol, Sorbitari Monolaurate, T-octylphenoxypolyethoxyethanol, Polysorbate 20, Polysorbate 40, Polysorbate 60 and Polysorbate 80, or a combination thereof.

6. The method of claim 5, wherein said non-ionic detergent or combination thereof is in the range of 0.1-10%.

7. The method of claim 2, wherein said lysis solution includes 1-10 M Guanidine HCl, 0.1-10% TWEEN™ 20 and 0.1-10% NP-40.

8. The method of claim 2, further comprising a phenol chloroform extraction step of said aqueous solution, before said mixing step b).

9. The method of claim 1, wherein the first mineral support and the second mineral support are porous or non-porous and comprised of metal oxides or mixed metal oxides, silica gel, silica membrane, glass particles, powdered glass, Quartz, Alumina, Zeolite, Titanium Dioxide, or Zirconium Dioxide.

10. The method of claim 1, wherein the first mineral support and the second mineral support are each silica membranes.

11. The method of claim 1, wherein said first or second dipolar aprotic solvent is selected from Acetone, Acetonitrile, Tetrahydrofuran (THF), Methyl Ethyl Ketone, N,N-Dimethylformamide (DMF), and Dimethyl Sulfoxide.

12. The method of claim 1, wherein both said first and second dipolar aprotic solvent are Acetone.

13. The method of claim 12, wherein in step a), said lower percent of acetone is about 35%, while in said step d), said higher percent of acetone is about 50%.

14. The method of claim 12, wherein yield of small RNA is at least 10 percent higher than the yield obtained using ethanol in the place of acetone.

15. The method of claim 2, wherein said biological sample is selected from cultured cells, microorganisms, plants and animals.

16. A method for separation and isolation of both large and small RNA from the same sample, comprising:
i) isolating small RNA following the steps of claim 1;
ii) optionally washing said first mineral support containing large RNA from step b) of claim 1; and
iii) eluting the large RNA from said first mineral support.

17. The method of claim 2, wherein both said first and second dipolar aprotic solvent are Acetone.

18. The method of claim 2, wherein in step a), said lower percent of acetone is about 35%, while in said step d), said higher percent of acetone is about 50%.

19. The method of claim 2, wherein yield of small RNA is at least 10 percent higher than the yield obtained using ethanol in the place of acetone.
